(19) United States
(12) Patent Application Publication
(10) Pub. No.: US 2013/0195820 A1
(43) Pub. Date: Aug. 1, 2013

(54) USE OF BLOOD GROUP STATUS III

(71) Applicants: Pirjo Wacklin, Helsinki (FI); Jaana Matto, Helsinki (FI); Harri Makivuokko, Kirkkonummi (FI); Jukka Partanen, Helsinki (FI); Janne Nikkila, Helsinki (FI)

(72) Inventors: Pirjo Wacklin, Helsinki (FI); Jaana Matto, Helsinki (FI); Harri Makivuokko, Kirkkonummi (FI); Jukka Partanen, Helsinki (FI); Janne Nikkila, Helsinki (FI)

(73) Assignee: SUOMEN PUNAINEN RISTI VERIPALVELU, Helsinki (FI)

(21) Appl. No.: 13/812,799

(22) PCT Filed: Jul. 26, 2011
(86) PCT No.: PCT/FI2011/050674

§ 371 Date: Apr. 5, 2013

(30) Foreign Application Priority Data
Jul. 26, 2010 (FI) 20105825

(51) Int. Cl. A61K 35/74

(52) U.S. Cl. CPC A61K 35/74
USPC 424/93.41; 435/34; 424/93.4; 435/252.7; 435/252.1

(57) ABSTRACT

The present invention relates to a microbial composition which is tailored based on the spectrum of microbes found more frequently from the intestine of non-secretor individuals than from the intestine of secretor individuals. The present invention further relates to a method of tailoring a microbial composition based on the spectrum of microbes found more frequently from the intestine of non-secretor individuals than from that of secretor blood group status. The present invention relates to use of the secretor status of an individual as a criterion for microbial supplementation tailored based on the differences in the spectra of microbes found between secretor and non-secretor individuals.

### FIELD OF THE INVENTION

The present invention relates to a microbial composition which is tailored based on the spectrum of microbes found more frequently from the intestine of non-secretor individuals than from the intestine of secretor individuals. The present invention further relates to a method of tailoring a microbial composition based on the spectrum of microbes found more frequently from the intestine of non-secretor individuals than from that of secretor blood group status. The present invention also relates to a microbial composition which is tailored based on the spectrum of microbes found more frequently from the intestine of secretor individuals than from the intestine of non-secretor individuals. The present invention further relates to a method of tailoring a microbial composition based on the spectrum of microbes found more frequently from the intestine of secretor individuals than from that of non-secretor blood group status. Further, the present invention relates to use of the secretor status of an individual as a criterion for microbial supplementation tailored based on the differences in the spectra of microbes found between secretor and non-secretor individuals. The present invention relates also to method of assessing the need of an individual for the tailored microbial supplementation by determining the secretory status of the individual. Also, the invention relates to the use of prebiotics, molecular compounds or additional supportive microbial strains, to increase the number of, and/or to augment the growth and/or functionality of these microbial strains.

### BACKGROUND OF THE INVENTION

Human intestinal tract is colonised with over 500 bacterial species, whose total number can exceed trillions of microbial cells in the colon. This microbiota in the large intestine is mainly composed of Firmicutes and Bacteroides phyla, which make up over 75% and 16% of total microbes in the gut (Eckburg et al., 2005, Science 308(5728):1635-8, Tap et al., 2009, Environ Microbiol 11(10):2574-84). Within Firmicutes phyla, Clostridium and its close relatives dominate with Clostridium leptum group (Clostridium cluster IV) and Eubacterium rectale-Clostridium coccoides group (Clostridium cluster XIVa) are the most prevalent groups (Tap et al. 2009). Bacteroides species found in the gut mainly belong to B. fragilis group. In spite of low diversity at the microbial phyla level, the gut microbiota composition among individuals is highly variable at species and strain level. In 17 human faecal samples, only 66 OTUs (“Operational Taxonomic Units”) of the 3180 detected were present in more than 50% of the individuals, creating so-called core microbiota (Tap et al. 2009). The core microbiota consisted mainly species of Bacteroides and clostridia; in addition, one Bifidobacterium spp and one Coprobacillus spp. were included in the core.

The microbiota has an important role in human health. It contributes to the maturation of the gut tissue, to host nutrition, pathogen resistance, epithelial cell proliferation, host energy metabolism and immune response (e.g. Dethlefsen et al., 2006, Trends Ecol Evol 21(9):517-23; Round and Mazmanian, 2009, Nat Rev Immunol 9(5):313-23). An altered composition and diversity of gut microbiota have been associated to several diseases (Round and Mazmanian, 2009), such as inflammatory bowel disease, IBD (Sokol et al., 2008, Proc Natl Acad Sci USA, 105(43):16731-6), irritable bowel syndrome (Mättö et al. 2005, FEMS Immunol Med Microbiol 43(2):213-22.), rheumatoid arthritis (Vaahtovuo et al., 2008, J Rheumatol 35(8):1500-5), atopic eczema (Kalliomaki and Isolauri. 2003 Curr Opin Allergy Clin Immunol 3: 15-20), asthma (Björksten 1999 Curr Drug Targets Inflamm Allergy 4: 599-604) and type 1 diabetes (Wen et al., 2008, Nature 455(7216):1109-13). Little is known, however, which species mediate beneficial responses. A decrease in the number of Faecalibacterium prausnitzii, a well-studied member of the C. leptum group, has been observed in IBD and evidence indicates that F. prausnitzii has anti-inflammaroty effects in vitro and in vivo (Sokol et al. 2008). Similarly, certain Bacteroides spp. strains have shown to possess therapeutic potential in prevention of colitis in mouse models (Round and Mazmanian, 2009).

The role of host genes on composition of gut microbes has been supported by twin studies, which showed that monozygotic twins have more similar gut microbiota than dizygotic twins or unrelated persons (Zoetendal et al., 2001, Microbial Ecology in Health and Disease 13(3):129-34). However, little is known which genes determine or regulate the microbial composition. Some gut microbes e.g. Helicobacter pylori and pathogenic species of bacteria and viruses have shown to use ABO blood group antigens as adhesion reseptors (Boren et al. Science 1993, 262, 1892-1895). Some microbes e.g. Bifidobacteria and Bacteroides thetaiotaomicron are also able to utilize blood group antigens or glycans found in ABO and Lewis antigens.

The ABO blood group antigens are not present in the mucus of all individuals. These individuals, said to have the ‘non-secretor’ blood group, do not have the functional FUT2 gene needed in the synthesis of secreted blood group antigens (Henry et al., Vox Sang 1995; 69(3):166-82). Hence, they do not have ABO antigens in their secretions and mucosa while those with blood group ‘secretor’ have the antigens. In most populations, the frequency of non-secretor individuals is substantially lower than that of secretor status; about 15-26% of Scandinavians are non-secretors (Eriksson et al. Ann Hum Biol. 1986 May-June; 13(3):273-85). The secretor/non-secretor status can be regarded as a normal blood group system and the phenotype can be determined using standard blood banking protocols (Henry et al. 1995). The genotype, that is, the major mutation in the FUT2 gene causing the non-secretor (NSS) phenotype in the European populations (Silva et al. Glycoconj 2010; 27:61-8) has been identified. Non-secretor phenotype has been demonstrated to be genetically associated for example, with an increased risk for Crohn's disease (McGovern et al. Hum Molec Genet 2010 Advance Access Published Jun. 22, 2010), with high vitamin B12 levels in the blood (Tanaka et al Am J Hum Genet 2009; 84:477-482), with resistance to Norovirus infection (Thorven et al J Virol 2005; 79: 15351-15355), with susceptibility to HI virus infection (Ali et al 2000, J Infect Dis 181: 737-739), with experimental vaginal candidiasis (Hurd and Domino Infection Immunit 2004; 72: 4279-4281), with an increased risk for asthma (Ronchetti et al. Eur Respir J 2001; 17: 1236-1238), with urinary tract infections (Sheinfeld et al N Engl J Med 1989; 320: 773-777), and with an animal hemorrhagic disease virus (Guillon et al. Glycobiology 2009; 19: 21-28).

The beneficial effects of certain microbial species/strains on maintaining or even improving of gut balance and growing evidence of their health effects on intestinal inflammatory diseases have caused a great interest on modulation of gut microbiota; and recently also on modulation of microbiota of other tissues such as oral, vaginal or skin. Gut microbiota can be modulated by taking probiotics, which currently belong mainly to Bifidobacteria and Lactobacillus genera.

Many probiotic supplements and products currently on the market are ineffective in promoting the desired health effects among most individuals. Thus, there is a continuous need for microbial and/or probiotic products that are able to mediate the health effects of the microbes more efficiently.

### BRIEF DESCRIPTION OF THE INVENTION

The present invention is based on the finding that individuals with non-secretor blood group status showed marked differences in their gut microbial composition in comparison to secretor individuals. Specifically, occurrence or abundance of certain Bacteroides and Clostridium leptum group genotypes, as defined using the method of Denaturating Gradient Gel Electrophoresis (DGGE), were higher in non-secretor individuals than secretor individuals.

The genotypes were:

band positions 25.30%, 26.40%, 39.00%, 42.40%, 47.00%, 50.40%, 56.80%, and 60.20% as defined by universal-DGGE analysis;

band positions 35.30%, 60.0%, and 69.00% as defined by Eubacterium rectale-Clostridium coccoides-group (EREC)-DGGE analysis;

band positions 4.80%, 10.20%, 23.80%, 36.00%, 38.70% and 41.10% as defined by Bacteroides-DGGE analysis; and

band positions 15.40%, 20.50%, 27.80%, 32.80%, 36.10%, 37.30%, 42.10%, 43.00%, 46.10%, 61.80%, 73.30%, 79.10%, 85.00% and 91.80% as defined by Clostridium leptum-DGGE.

Further, the present invention is based on the finding that individuals with secretor blood group status showed higher occurrence or abundance of the following genotypes in their microbiota:

band position 62.60% as defined by Eubacterium rectale-Clostridium coccoides-group (EREC)-DGGE analysis; and

band position 56.20% as defined by Clostridium leptum-DGGE analysis.

In addition, using the Human Intestinal Tract (HIT) chip hybridisation and high throughput genomic sequencing of bacterial 16S rRNA gene, it was possible to demonstrate that the microbiota were different between non-secretors and secretors.

Thus, the non-secretor blood group status was found to be a host genotype, which determines the composition of intestinal microbes in man. This finding can be used as a basis for targeted modulation of intestinal microbial population tailored according to non-secretor/secretor status of an individual. The present invention can be targeted to stabilisation of the gut microbiota of an individual using those bacteria that were found to be typical to individuals with the same secretor/non-secretor phenotype as the individual to be treated or a bacterial product enriched with those bacteria that were found to be typical to individuals with the same secretor/non-secretor phenotype as the individual to be treated. The stabilisation can be either prophylactic, i.e. started before treatments disturbing the balance of gut microbiota, or it can be started once the symptoms develop. Further, the present invention can be targeted to increasing the number of those beneficial bacteria scarcely found in individuals with the same secretor/non-secretor phenotype as the individual to be treated by administering the said bacteria to the individual. The invention describes which particular microbes should be enriched in a microbial and/or probiotic supplement or composition to improve the responsiveness and/or effect of the product. This tailoring or optimising or potentiating can be done to an existing microbial, probiotic and/or synbiotic product, or to a microbial strain not currently used as a probiotic. Moreover, the tailoring can be done by applying fecal transplantion with a fecal microbiota inoculum prepared from fecal material obtained from an individual representing the same secretor group as the fecal transplant donor (balancing of disturbed microbiota) or from an individual representing a different secretor group than the fecal transplant donor i.e., for increasing the richness of the microbiota in cases where the diversity of dominant microbiota is reduced.

Accordingly, an object of the present invention is a nonsecretor/secretor genotype based microbial composition which is tailored based on the spectrum of bacteria found in the mucosal tissue of at least one individual with non-secretor or secretor blood group phenotype. Another object of the present invention is a microbial composition which comprises at least one of the strains having any of the following genotypes:

band position 25.30%, 26.40%, 39.00%, 42.40%, 47.00%, 50.40%, 56.80% or 60.20% as defined by universal-DGGE analysis; or

band position 35.30%, 60.00%, 62.60% or 69.00% as defined by Eubacterium rectale-Clostridium coccoides-group (EREC)-DGGE analysis; or

band position 4.80%, 10.20%, 23.80%, 36.00% 38.70% or 41.10% as defined by Bacteroides-DGGE analysis; or

band position 15.40%, 20.50%, 27.80%, 32.80%, 36.10%, 37.30%, 42.10%, 43.00%, 46.10%, 56.20% or 61.80%, 73.30%, 79.10%, 85.00% or 91.80%, as defined by Clostridium leptum-DGGE analysis.

A further object of the present invention is a microbial composition which is tailored based on the spectrum of microbes found more frequently from the intestine of the non-secretor individuals than from the intestine of secretor individuals. An even further object of the present invention is a microbial composition which is tailored based on the spectrum of microbes found more frequently from the intestine of the secretor individuals than from the intestine of non-secretor individuals. Further, an object of the present invention is a method of tailoring a microbial composition based on the spectrum of bacteria found in the mucosal tissue of at least one individual with non-secretor or secretor blood group phenotype. Another object of the invention is use of the secretor blood group status of an individual in assessing the need for tailored microbial supplementation, i.e., as a criterion for microbial supplementation tailored based on the differences in the spectra of microbes found between secretor and non-secretor individuals. The present invention relates also to method of assessing the need of an individual for microbial supplementation by determining the secretory status of the individual. Also, an object of the invention is the use of prebiotics, molecular compounds or additional supportive microbial strains, to increase the number of, and/or to augment the growth and/or functionality of microbes in the intestine.

A further object of the present invention is a use of the secretor blood group status of an individual in estimating a dose of microbial supplementation needed for a desired effect.

The objects of the invention are achieved by the compositions, methods and uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows the RDA plot of HITChip analysis based on data hybridisation signals of species-like (level 1) bacterial groups of Example 8.

### DETAILED DESCRIPTION OF THE INVENTION

The present invention is based on the finding that a blood group system, secretor/non-secretor status, determines the spectrum or composition of microbial species and/or strains found in the human mucosal tissues, especially in the intestine. Individuals with non-secretor blood group status had marked differences in their gut microbial composition as compared to individuals with secretor status. According to the present invention, the blood group system secretor/non-secretor is a major genetic factor in the host determining the variation in the microbiota. The secretor/non-secretor status can be regarded as a normal blood group system and the phenotype can be determined using standard blood banking protocols. The genotype, that is, the mutation in the FUT2 gene causing the non-secterot (NSS) phenotype can be detected by various standard DNA-based techniques, such as allele-specific PCR amplification, sequencing, or using oligonucleotide probes, well-known in the art. The gut microbiota has an important role in human health; importantly, an altered composition and/or altered diversity of gut microbiota have been associated to several diseases.

According to the present invention, occurrence or abundance (i.e. band intensity) of certain genotypes of Eubacterium rectale-Clostridium coccoides, Bacteroides and Clostridium leptum group were higher in non-secretor individuals than in secretor individuals. The significant difference in gut microbiota between non-secretors and secretors was also demonstrated by HIT chip hybridisation and sequencing of bacterial 16S rRNA gene.

Denaturating Gradient Gel Electrophoresis, DGGE, is a method of choice to detect differences in spectrum or abundance of different bacterial genotypes. The method is well described in the art (Vanhoutte et al. FEMS Microb Ecol 2004; 48; 437-446; Matsuki et al. Applied and Environmental Microbiology 2004; 70; 7220-7228; Satokari et al. AEM 2001; 67: 504-513; Mättö et al. FEMS Immunol Med Microbiol. 2005; 43: 213-22). In the method, specific PCR primers are designed so that in each experimental setting, only the desired bacterial group or groups are analysed. The differences in band positions and/or their occurrence and/or intensity indicate differences in bacterial compositions between faecal samples. Base composition of the PCR amplified fragment determinates the melting and, thus the mobility of the fragment in the denaturing gradient in gel. The final position of the fragment in gel is consequently specified by the DNA sequence of the fragment, the applied denaturing gradient and the electrophoresis running conditions. The optimised running conditions and denaturing gradient of the gels for the bacterial groups used in this invention are shown in Table 2. The position of each fragment, the “band position”, between different gel runs are normalised by using standards. The band position is indicated relative to length of the gel, the top being 0% and the bottom edge being 100%. The standards used were composed of PCR amplifled fragments of the relevant strains belonging to each bacterial group as described in Table 2.

The term bacterial genotype refers to those strains having the same “band position” in the relevant DGGE analysis. Each genotype or a group of closely-related genotypes can be presented as a “band position”. In the present invention, each band position refers to the band positions of the given %-value +/−1% unit, i.e. 25.30% refers to any value between 24.30% and 26.30%, when analysed using the methodology described above. It is noted than depending on the exact conditions the nominant %-value can vary; the relative position of the band to the relevant standard is important.

According to the invention, the following bacterial genotypes had a higher abundance and/or higher band intensity in the gut microbiota of non-secretors than in that of secretors:

band positions 25.30%, 26.40%, 39.00%, 42.40%, 47.00%, 50.40%, 56.80% and 60.20% as defined by universal-DGGE analysis;

band positions 35.30%, 60.00% and 69.00% as defined by Eubacterium rectale-Clostridium coccoides-group (EREC)-DGGE analysis;

band positions 4.80%, 10.20%, 23.80%, 36.00%, 38.70% and 41.10% as defined by Bacteroides-DGGE analysis; and

band positions 15.40%, 20.50%, 27.80%, 32.80%, 36.10%, 37.30%, 42.10%, 43.00%, 46.10%, 61.80%, 73.30%, 79.10%, 85.00% and 91.80% as defined by Clostridium leptum-DGGE analysis.

In addition, the following microbial genotypes had a higher frequency or occurrence in samples from non-secretors than from secretors:

band position 56.80% as defined by universal-DGGE analysis; band position 60.0% as defined by Eubacterium rectale-Clostridium coccoides-group (EREC)-DGGE analysis; and

band position 23.80% as defined by Bacteroides-DGGE analysis.

In addition, the following genotypes had higher adundance and/or band intensity in gut microbiota of secretors than in that of non-secretors:

band position 62.60% as defined by Eubacterium rectale-Clostridium coccoides-group (EREC)-DGGE analysis; and

band position 56.20% as defined by Clostridium leptum-DGGE analysis.

The above mentioned genotypes are examples of genotypes here referred to as “genotypes typical to individuals” with secretor or non-secretor phenotype. It is of note that as the secretor/non-secretor trait, that is the expression of ABO structures in mucosa, can be identified in all mucosal tissues, the invention is relevant to all mucosal tissues of an individual and not restricted to the gut or faecal samples.

It is possible to isolate the bacterium or strain representing a particular PCR-DGGE genotype. A faecal sample can be cultured on non-selective or selective culture medium in the conditions supporting the growth of the targeted bacterial group. For example, Brucella blood agar (BRU) or reinforced clostridial agar with chinablue and horse blood (RCBA) can be used for anaerobic bacteria such as clostridia and their close relatives. In addition several selective culture media e.g. Bacteroides Bile esculin (BBE) for the isolation of Bacteroides spp. and its close relatives, Beerens and Raffinose bifidobacterium (RB) for bifidobacteria, and Rogosa and LAMVAB for lactobacilli, respectively, can be used. The plates are incubated in conditions supporting the growth of the targeted bacteria e.g. anaerobiosis at 37 C. Bacterial isolates can be sub-cultured from the culture plates and identified to the species level by 16S rDNA sequencing. An isolate is then run in DGGE with known control samples with a particular DGGE band position.

Steps for tailoring a microbial composition typically comprise:
determination of secretor/non-secretor status of an individual using standard methods described in the art;
determination of spectra of microbes typical to the secretor and/or non-secretor phenotypes by determining the microbes found in the intestinal samples of a sufficient number (at least one) of individuals with secretor and/or non-secretor phenotypes, based on the analyses using DGGE and optionally sequencing the 16S rRNA gene and isolating the relevant strains;
determination of bacteria or strains or bacterial genotypes significantly enriched in samples from secretor or non-secretor individuals by comparing the spectra of microbes between secretor and non-secretor samples;
optionally preparing a bacterial product containing a high amount of the bacteria and/or strains or bacterial genotypes significantly enriched in the desired secretor/non-secretor samples.

Then microbiota of a recipient can be stabilized and/or modified by administering the secretor/non-secretor tailored bacterial product to the recipient.

The culturing can also be performed in a device mimicking the gastrointestinal tract. One such is the TNO TIM-1 model. Typically, faecal slurries acquired by mixing faeces with artificial saliva and sterile water are used as an input for the TIM-1 model. The faecal slurries can be prepared from study groups, for example, from pooled non-secretor and secretor samples. In TNO TIM-1 model, various parameters can be adjusted, e.g. level of gastric secretion, time to addition of bile and pancreatin, etc. In each compartment the physiological concentrations of bile salts, pancreatic enzymes and electrolytes simulates an average physiological passage through the small intestine. The survival of targeted bacteria can be compared between the study groups, in order to look for functional differences between bacterial populations.

The microbiota composition can also be prepared by using faecal material as an inoculum. The faecal slurry can be prepared either by direct dilution to an appropriate diluent or by separation of selected bacterial groups from the sample.

The present invention provides means for the use of secretor status for tailoring probiotic supplements optimized according to non-secretor (NSS) and secretor (SS) genotype of the host. Optimization is based on the rationale that according to the present invention, certain bacterial genotypes are essentially missing or their proportion of the entire gut microbiota is lower in an individual or host having secretor genotype than in non-secretor genotype. Further, the optimization is based on the rationale that according to the present invention, certain bacterial genotypes are essentially missing or their proportion of the entire gut microbiota is lower in an individual or host having non-secretor genotype than in secretor genotype. The probiotic preparation or product can be tailored so that it contains higher amounts or proportions of those bacterial genotypes or strains that are known to have altered abundances and whose increase in number is desired. For stabilisation of disturbed microbiota, the preparation can be tailored so that it contains high amounts or proportions of those bacterial genotypes or strains that are known, according to the present invention, to be typical to the secretor/non-secretor status of the individual to be treated.

In one embodiment of the invention, the non-secretor/secretor genotype based microbial composition is tailored based on the spectrum of bacteria found in the mucosal tissue of at least one individual with non-secretor or secretor blood group phenotype.

In an embodiment of the invention, the microbial composition comprises at least one of the strains having any of the following genotypes:

band position 25.30%, 26.40%, 39.00%, 42.40%, 47.00%, 50.40%, 56.80% or 60.20% as defined by universal-DGGE analysis; or

band positions 35.30%, 60.00%, 62.60% or 69.00% as defined by Eubacterium rectale-Clostridium coccoides-group (EREC)-DGGE analysis; or

band positions 4.80%, 10.20%, 23.80%, 36.00%, 38.70% or 41.10% as defined by Bacteroides-DGGE analysis; or

band position 15.40%, 20.50%, 27.80%, 32.80%, 36.10%, 37.30%, 42.10%, 43.00%, 46.10%, 56.20%, 61.80%, 73.30%, 79.10%, 85.00% or 91.80%, as defined by Clostridium leptum-DGGE analysis.

In another embodiment, the microbial composition comprises two or more of the strains specified above. In a further embodiment, the microbial strains belong to the Clostridium leptum group.

In an embodiment of the invention, the microbial composition comprises at least one of the strains having any of the following genotypes:

band position 25.30%, 26.40%, 39.00%, 42.40%, 47.00%, 50.40%, 56.80% or 60.20% as defined by universal-DGGE analysis; or

band position 35.30%, 60.00%, or 69.00% as defined by Eubacterium rectale-Clostridium coccoides-group (EREC)-DGGE analysis; or

band position 4.80%, 10.20%, 23.80%, 36.00%, 38.70% or 41.10% as defined by Bacteroides-DGGE analysis; or

band position 15.40%, 20.50%, 27.80%, 32.80%, 36.10%, 37.30%, 42.10%, 43.00%, 46.10%, 61.80%, 73.30%, 79.10%, 85.00% or 91.80% as defined by Clostridium leptum-DGGE analysis.

In another embodiment, the microbial composition comprises two or more of the strains specified above. In a further embodiment, the microbial composition comprises at least one of the strains defined above by universal-DGGE analysis, at least one of the strains defined above by EREC-DGGE analysis, at least one of the strains defined above by Bacteroides-DGGE analysis and at least one of the strains defined above by Clostridium leptum-DGGE analysis. In a further embodiment, the microbial composition comprises all the strains defined above. In an even further embodiment, the microbial composition comprises the strains defined above by universal-DGGE analysis or the strains defined above by EREC-DGGE analysis or the strains defined above by Bacteroides-DGGE analysis or the strains defined above by Clostridium leptum-DGGE analysis.

In one embodiment of the invention, the microbial composition comprises at least one of the strains having any of the following genotypes: band position 62.60% as defined by Eubacterium rectale-Clostridium coccoides-group (EREC)-DGGE analysis; or

band position 56.20% as defined by Clostridium leptum-DGGE analysis.

In a further embodiment, the microbial composition comprises the strains having any of the following genotypes:

band position 62.60% as defined by Eubacterium rectale-Clostridium coccoides-group (EREC)-DGGE analysis; and

band position 56.20% as defined by Clostridium leptum-DGGE analysis.

In an even further embodiment of the present invention, the microbial composition is tailored based on the spectrum of non-bifidobacterial bacteria found in the mucosal tissue of at least one individual with non-secretor or secretor blood group phenotype.

The present invention can be targeted to stabilisation of the gut microbiota of an individual using those bacteria that were found to be typical to individuals with the same secretor/non-secretor phenotype as the individual to be treated or a bacterial product enriched with those bacteria that were found to be typical to individuals with the same secretor/non-secretor phenotype as the individual to be treated. The stabilisation can be either prophylactic, i.e. started before treatments disturbing the balance of gut microbiota, or it can be started once the symptoms develop. Further, the present invention can be targeted to increasing the number of those beneficial bacteria scarcely found in individuals with the same secretor/non-secretor phenotype as the individual to be treated by administering said bacteria or adding the said bacteria into a product.

The microbial preparation according to the present invention is targeted, for example, to a relief of symptoms and/or to the therapy of diseases in which gut microbiota plays an important role, such as inflammatory bowel disease, IBD (Sokol et al. 2008.), irritable bowel syndrome (Mättö et al. 2005.), rheumatoid arthritis (Vaahtovuo et al. 2008), atopic eczema (Kalliomäki et al. 2003), asthma (Björksten, 1999) and type 1 diabetes (Wen et al. 2008). In additiont, the target is general immunomodulation, for example, induction of regulatory T lymphocytes (Round and Mazmanian PNAS doi/10.1073/pnas.0909122107), which are known to induce immunotolerance in organ and stem cell transplantations or to suppress the immune response.

In one embodiment, the invention is related to a probiotic composition for prevention or treatment of inflammatory bowel disease (IBD). IBD is an excellent target disease for the invention as an altered microbiota composition in the patients has been reported (Sokol et al. Inflamm Bowel Dis. 2006 February; 12(2):106-11). Furthermore, it is established (McGovern et al. Hum Molec Genet 2010; 19(17): 3468-76) that the non-secretor phenotype, i.e. FUT2 gene defect, confers genetic susceptibility to IBD. Hence, it is plausible that the composition according to the present invention is particularly effective in IBD. The treatment can be targeted to a relief of the symptoms and/or to prevention of relapses and/or to increasing the overall quality of life in IBD. It also may be administered together with other currently known drugs for IBD. In an embodiment, the composition of the present invention is targeted to those IBD patients with the non-secretor phenotype.

In another embodiment, the invention is related to a probiotic composition for prevention or treatment of microbial infections i.e. diarrhoea and respiratory tract infections as also in these indications therapeutic potential of probiotics (Chouraqui et al. J Pediatr Gastroenterol Nutr. 2004 March; 38(3):242-3; de Vrese et al. Clin Nutr. 2005 August; 24(4):479-80), and an increased frequency in non-secretor individuals (Ahmed et al. 2009 Infect Immun. 2009 77(5):2059-64; Raza et al. BMJ. 1991, 303(6806):815-8) have been described.

In a further embodiment, the invention is related to a probiotic composition for prevention or treatment of irritable bowel syndrome as disturbed microbiota (Mättö et al. 2005) and potential of probiotic products have been described in IBS (Kajander et al. Aliment Pharmacol Ther. 2008 27(1):48-57).

In yet another embodiment, the invention is related to a probiotic composition for prevention or treatment of allergy/atopy in children. It is established that babies who develop allergy have disturbed microbiota in their intestine during the first year of life (Björkstén et al. J Allergy Clin Immunol. 2001 108(4):516-20). Moreover, it has been shown that bacterial composition in the milk of allergic mothers differs from that of non-allergic mothers (Grönlund et al. Clin Exp Allergy. 2007, 37(12):1764-72). Probiotic products have shown potential in prevention of atopic eczema (Yoo et al. Proc Am Thorac Soc (2007) 4, 277-282).

In one embodiment of the invention, the microbial and/or probiotic composition or the supplement comprising the composition is particularly suitable and effective, in use for the enhancement of the diversity and numbers of intestinal bacteria, or balancing the microbiota in an individual suffering from celiac disease. It has recently been demonstrated that patients with celiac disease and its different clinical forms have disturbed gut microbiota (Wacklin P, Pusa E, Kaukinen K, Maki M, Partanen J, and Mättö J. Composition of the mucosa-associated microbiota in the small intestine of coeliac disease patients and controls. A poster presented in the Rowett-INRA Gut Microbiologyconference, Aberdeen, UK, 22-25.6. 2010). Briefly, to evaluate differences between disease symptom groups, intestinal microbiota compositions were assessed from mucosal biopsy samples from 26 coeliac disease patients (further sub-grouped to gastrointestinal, anemia and dermatitis herpetiformis symptom groups) and 25 healthy controls. The samples were analysed by PCR-DGGE with universal bacterial primers. Intestinal microbiota of the coeliac disease patients representing different symptom groups clustered in clearly distinct groups. The finding indicates that the microbiota composition is variable between individuals and in intestinal disorders disease-group related differences in the microbiota composition exist, which should be taken into consideration in applications targeting for balancing or modulating the intestinal microbiota of individuals suffering from immunological gastrointestinal disorders.

In another embodiment of the invention, the secretor/non-secretor status can be used to augment the stabilisation of mucosal microbiota composition of an individual after disorders or treatments known to disturb the balance of mucosal microbiota. Examples of these comprise treatments with broad spectrum antibiotics, irradiation or cytotoxic therapies related to cancer treatments or bone marrow transplantation or its complications such as graft-versus-host disease and/or gastroenterological infections by e.g. Noro-virus or Helicobacter.

The disturbed balance of the gut microbiota in patients who have received bone marrow transplantantation has recently been demonstrated (Pusa E, Taskinen M, Lahteenmaki K, Kaartinen T, Partanen J, Vettenranta K, Mättö J. Do intestinal bacteria or donor derived responsiveness to microbial stimuli play a role post allogeneic HSCT?, A poster presented in the 7^{th} Meeting of the EBMT Paediatric Diseases Working Party, 2-4 June, 2010 Helsinki, Finland). To evaluate the intestinal microbiota disturbance following chemotherapy or irradiation treatments related to treatments of malignant diseases intestinal microbiota composition of hematopoietic transplantation patients was monitored. Faecal samples were collected from pediatric HSCT patients both before transplantation and at different time points, up to 6 months, after the transplantation and their donors. Microbiota profiling was performed by applying standard PCR-DGGE. PCR-DGGE analysis revealed remarkable instability of the intestinal microbiota after transplantation. The similarity of the dominant microbiota was extremely low during the first month after transplantation while up to 94% similarity was detected between the samples obtained 4-6 months from the transplantation. PCR-DGGE specific bacterial group targeted primers revealed absense of several common intestinal bacteria (e.g. bifidobacteria, lactobacilli, C. leptum group) in several samples obtained within one month from transplantation. These findings indicate a drastic disturbance of the intestinal microbiota during HSCT and a need for targeted microbiota modulation in these patients.

The present invention is further targeted to treatment of diseases or traits, having the FUT2 gene (i.e. the secretor blood group status) as a genetic susceptibility factor. These comprise, just to give examples, low levels of vitamin B12 in the blood, various clinical forms of inflammatory bowel disease, urinary tract infections, vaginal candidiasis, Noro- and HI-virus infections and infections by hemorrhagic viruses. It is likely, due to the crucial role of FUT2 in modulating the microbiota, that a higher number of diseases will be identified in the future by screening the FUT2 locus. Probiotic treatments typically are used to direct or change the microbiological balance in the gut toward healthier one, or toward the microbial spectrum “typical to individuals” with the non-susceptible FUT2 genotype. The present invention is particularly related to treatments directed to individuals with the non-secretor status. Individuals with the non-secretor phenotype typically require higher dosages and/or preparations with more diverse microbial strains than secretors. Thus, the present invention relates also to use of the secretor/non-secretor status of an individual to augment the stabilisation of mucosal microbiota composition in disorders related to, or after treatments leading to unbalance of mucosal microbiota.

The present invention also relates to a method of identifying an individual at risk for suffering from a disorder related to unbalance of mucosal microbiota, such as a gastrointestinal disorder by determining the secretory status of said individual.

The present invention further relates to a use of the secretor/non-secretor status of an individual in estimating a dose of bacterial supplementation needed for a desired effect.

In one embodiment, the microbial preparation is not orally administered but is a solution or ‘salva’ which is directly administered onto the target mucosal tissue. Examples of this embodiment are disorders of gingival or vaginal tissues.

In one embodiment, the invention is related to microbial or probiotic composition targeted to elderly individuals for supporting the maintenance of balanced microbiota in the gut and/or some other mucosal, such as oral, vaginal or skin tissue. In another embodiment, the invention is related to microbial or probiotic composition targeted to infants for stabilisation of the microbiota in the gut and/or some other mucosal tissue. Limited repertoire of commensal microbes typical to infants confers them susceptible for infections; optimisation of the composition according to the present invention increases the efficacy of the treatment. The treatment can be either prophylactic before an infection for individuals, e.g. elderly or infants, with a high infection risk (i.e, probiotic type), or therapeutic during the infection.

The present invention also provides means for improving responsiveness and/or effect of the microbial and/or probiotic product. Not all individuals are responsive for current probiotic products; a tailored composition enriched with microbial strains which according to the present invention have a better ability to stay alive and grow in the gut or other mucosal tissue improves responsiveness.

Severe disturbances in the mucosal or gut microbiota can be a result of treatments related to e.g. cancer therapy, haematopoietic stem cell transplantation or its complications such as graft-versus-host disease, or use of antibiotics. The present invention relates to the use of secretor/non-secretor status in estimating the most effective way for stabilisation of the microbiota. Stabilisation can be achieved most effectively by probiotic products tailored according to the present invention.

The present invention provides a novel and effective method for screening and identification of novel probiotic strains. In one embodiment, the NSS/SS genotype forms the basis for the selection of the most efficient source of the faecal samples, the starting point for identification of suitable probiotics. Faecal samples from individuals with non-secretor status can be used for isolating efficiently those bacterial strains more abundant in non-secretor genotype. The fact that these strains, e.g. those belonging to C. leptum or B. fragilis group, are frequent in the microbiota of hosts with NSS genotype indicates that they obviously are particularly viable in the gut of NSS hosts. A good colonization ability and viability in the gut are essential features for a probiotic. The invention can be applied in the similar way when other mucosal tissues than the gut are considered as a target. The term “mucosal tissue” here refers to orogastrointestinal, gut, skin, oral, respiratory, and/or uro-genital tissues or samples derived from these or to any of the other indications described above.

In one embodiment of the present invention, determination of the secretor/non-secretor status and use of the result to consequently predict the bacterial spectrum of an individual is used to optimize faecal transplantation. This can be done as the only test or in combination with an actual analysis of microbiota composition. The result can be used as a criterion for choosing a donor for faecal transplantation. Bacteria derived from the faecal transplant from a donor representing the same secretor/non-secretor type with the recipient are likely to have a better colonisation ability and efficacy than those derived from a mismatched donor. Faecal transplantation can be used for a therapy in severe Clostridium difficile infections (MacConnachie et al. QJM 2009, 102(11), 781-4); the present invention can improve the efficacy of the treatment. The efficacy can be further improved by giving a secretor/non-secretor matched bacterial preparation post-transplantation in order to improve the stabilisation of the gut microbiota of the recipient. The preparation can contain the spectrum of bacteria found commonly in samples classified according to sectretor/non-secretor status and can be produced e.g. as a fresh, frozen pellet or freeze-dried product formulation. In addition to Clostridium difficile infection, faecal transplantation once optimised according to the present invention can be used to stabilise gut microbiota in many other disorders related to or resulting to severe disturbances in gut microbiota, for example, diseases requiring intensive antibiotic treatments, chemotherapy or total body irradiation before bone marrow transplantation.

In an embodiment, the secretor/non-secretor status is used, optionally together with standard analyses of microbial composition in a sample, in estimating whether microbial composition in a particular mucosal tissue, such as the gut of an individual is in balance. The secretor/non-secretor status or genotype can be determined in vitro from the blood or saliva sample of the host and the microbial composition from the mucosal or faecal samples using standard methods, well known in the art. The microbiota composition of an individual so obtained can be compared to the reference secretor/non-secretor specific compositions that can be obtained by determining the microbiota compositions in a number of samples from healthy individuals whose secretor/non-secretor status are known. The secretor/non-secretor specific compositions can be obtained by identifying the bacterial strains and/or species or genotypes enriched in the secretor samples or in the non-secretor samples. Host secretor/non-secretor genotype together with the standard analysis of microbial spectrum, provides a more reliable estimate of the balance than the analysis of the mucosal or faecal sample alone, because the genotype partially determines the assumed, normal composition. This result can be used to estimate the need by an individual for probiotic supplementation in disorders assumed or known to be related to variation in the microbiota. The result can also be used to reduce infection risk. Non-secretors are known to be more vulnerable to infections (Blackwell, C. C. 1989. FEMS Microbiology Immunology 47, 341-350). A balanced and diverse population of beneficial commensal gut microbes, achieved or augmented by probiotics tailored according to the present invention, is therefore particularly important for non-secretors.

Thus, in one embodiment, the present invention relates to a method for determining the balance of gut microbiota of an individual wherein the method comprises;
determining secretor/non-secretor genotype of an individual,
determining the composition of gut microbiota of the said individual,
comparing the composition of the gut microbiota of the said individual to the typical composition of gut microbiota according to the secretor/non-secretor genotype.

The term ‘probiotic’ here refers to any bacterial species, strain or their combinations, with health supportive effects, not limited to currently accepted strains or to intestinal effects. The probiotic as defined here may be applied also by other routes than by ingestion, e.g. by applying directly to desired tissue.

The term ‘prebiotic’ here refers to any compound, nutrient, or additional microbe applied as a single additive or as a mixture, together with probiotics or without probiotics, in order to augment a desired probiotic health effect or to stimulate the growth and activity of those microbes in the mucous tissue, such as digestive system, which are assumed to be beneficial to the health of the host body.

The terms “microbial” and “bacterial” here are used as synonyms and refer to any bacterial or other microbial species, strains or their combinations, with health supportive effects, not limited to strains currently accepted as probiotics.

The terms “microbial composition or microbial product” here refer to a microbial preparation and a probiotic or prebiotic product, including those applied by other routes than the traditional ingested probiotic, e.g. applied directly onto mucosal tissues such as skin or uro-genital tract, or a product for faecal transplant.

The term “tailoring” refers to determining the secretor/non-secretor blood type of the recipient and the typical and/or characteristic mucosal bacterial repertoire of the secretor/non-secretor blood type by methods known in the art and optionally manufacturing a bacterial composition based on the determined bacterial repertoire. Alternatively, it refers to determining the typical and/or characteristic mucosal bacterial repertoire of the secretor or non-secretor blood type and the secretor/non-secretor blood type of the recipient by methods known in the art and optionally manufacturing a bacterial composition based on the determined bacterial repertoire. After this tailoring step the bacteria or the bacterial composition is administered to the recipient.

The probiotic compositions and supplements so designed may have beneficial effects on the health and/or well-being of a human and may be formulated into a functional food product or a nutritional supplement as well as a capsule, emulsion, or powder.

A typical probiotic ingredient is freeze-dried powder containing typically 10¹⁹-10¹² viable probiotic bacterial cells per gram. In addition it normally contains freeze drying carriers such as skim milk, short sugars (oligosaccharides such as sucrose or trehalose). Alternatively, the culture preparation can be encapsulated by using e.g. alginate, starch, xanthan as a carrier. A typical probiotic supplement or capsule preparation contains approximately 10⁹-10¹¹ viable probiotic bacterial cells per capsule as a single strain or multi-strain combination.

A typical probiotic food product, which can be among others fermented milk product or juice, contains approximately 10⁹-10¹¹ viable probiotic bacterial cells per daily dose. Probiotics are incorporated in the product as a probiotic ingredient (frozen pellets or freeze dried powder) or they are cultured in the product during fermentation.

The invention will be described in more detail by means of the following examples. The examples are not to be construed to limit the claims in any manner whatsoever.

### EXAMPLES

### Materials and Methods

The materials and methods described herein are common to examples 1 to 5.

59 healthy adult volunteers (52 females and 7 males) were recruited to the study. Both faecal and blood samples were collected from 59 volunteers. The age of the volunteers ranged from 31 to 61 and was in average 45 years.

Faecal samples were frozen within 5 hours from defecation. DNA from 0.3 g of faecal material was extracted by using the FASTDNA® SPIN KIT FOR SOIL (Qbiogene).

PCR-DGGE methods were optimised to detect dominant eubacteria (universal group), Eubacterium rectale-Clostridium coccoides (EREC) group, Bacteroides fragilis group, Clostridium leptum group. Partial eubacterial 16S rRNA gene was amplified by PCR with group specific primers (shown in table 1). Amplified PCR fragments were separated in 8% DGGE gel with denaturing gradient ranging from 45% to 60%. DGGE gels were run at 70 V for 960 mins. DGGE gels were stained with SYRBSafe for 30 mins and documented with Safelmager Bluelight table (Invitrogen) and Aplhalmager HP (Kodak) imaging system.

Digitalised DGGE gel images were imported to the Bionumericsprogram version 5.0 (Applied Maths) for normalisation and band detection. The bands were normalised in relation to a marker sample specific for the said bacterial groups. Band search and bandmatching was performed as implemented in the Bionumerics. Bands and bandmatching were manually checked and corrected. Principal component analysis was calculated in Bionumerics. Other statistical analyses (Anova, Kruskal-Wallis test and Fisher exact test) were computed with statistical programming language R, version 2.8.1.

The bands were excised from DGGE gels. DNA fragments from bands were eluted by incubating the gel slices in 50 μl of sterile H₂O at +4° C. overnight. The correct positions and purity of the bands were checked for each excised bands by amplifying DNA in bands and re-running the amplified fragments along with the original samples in DGGE. Bands which produced single bands only and were in the correct position in the gels were sequenced. The sequences were trimmed, and manually checked and aligned by ClustalW. The closest relatives of the sequences were searched using Blast and NCBI nr database. Distance matrix of the aligned sequences was used to compare the similarity of the sequences.

TABLE 1
Primers and their sequences used in this study

| Target group | Primer | Sequence* | Reference** |
|---|---|---|---|
| Universal | U-968-F-GC | GCglamp1-AACGCGAAGAACCTTA | Nübel et al. 1996 |
| Universal | U-1401-R | CGGTGTGTACAAGACCC | Nübel et al. 1996 |
| Lactobacillus | Lac1 | AGCAGTAGGGAATCTTCCA | Walter et al. 2001 |
| Lactobacillus | Lac2GC | GCglamp2-ATTYCACCGCTACACATG | Walter et al. 2001 |
| EREC | CcocF | AAATGACGGTACCTGACTAA | Matsuki et al. 2002 |
| EREC | CcocR-GC | GCglamp1-CTTTGAGTTTCATTCTTGCGAA | Maukonen et al. 2006 |
| B. fragilis | BfraF | ATAGCCTTTCGAAAGRAAGAT | Matsuki et al. 2002 |
| B. fragilis | BfraR+GC | GCglamp1-CCAGTATCAACTGCAATTTTA | Matsuki et al. 2002 |
| C. leptum | Clept-F | GCACAAGCAGTGGAGT | Matsuki et al. 2004 |
| C. leptum | CleptR3-GC | GCglamp1-CTTCCTCCGTTTTGTCAA | Matsuki et al. 2004 |

*GCglamp1 sequence: CGCCCGGGGCGCGCCCCGGGCGGGGCGGGGGCACGGGGGG GCglamp2 sequence: CGCCCGCCGCGCCCCGCGCCCGGCCCGCCGCCCCCGCCCC
**References: Nübel et al. 1996 J Bacteriol. 178: 5636-43. Walter et al. 2001 Appl Environ Microbiol. 67: 2578-2585. Matsuki 2002 Appl Environ Microbiol. 68: 5445-51. Matsuki 2004 Appl Environ Microbiol. 70: 7220-8. Maukonen 2006. FEMS Microbiol Ecol. 58: 517-28.

TABLE 2
The optimised DGGE gel gradients, electrophoresis running
conditions for the each studied bacterial group and strains used in the
standards

| | | | Electrophoretic running | |
|---|---|---|---|---|
| Bacterial | | DGGE gel | conditions in Dcode system | |
| group | primers* | gradient | (Bio-Rad) | Strains in standard |
| Universal | U968F-GC, | 38-60% | 70 V, 960 mins | A. cacae DSM 14662 |
| | U1401 R | | | C. perfringens DSM 756 |
| | | | | E. ramulus DSM 15687 |
| | | | | F. prausnitzii DSM 17677 |
| | | | | E. coli DSM 30083 |
| | | | | L. rhamnosus DSM 96666 |
| | | | | P. melaninogenica DSM 7089 |
| Bifido- | Bif164F, | 45-60% | 70 V, 960 mins | B. adolescentis DSM 981074 |
| bacterium | Bif662R-GC | | | B. angulatum DSM 20098 |
| | | | | B. longum DSM 96664 |
| | | | | B. catenulatum DSM 16992 |
| | | | | B. lactis DSM 97847 |
| Lacto- | Lac1, | 38-55% | 70 V, 960 mins | L. plantarum E-79098 |
| bacillus | Lac2-GC | | | L. cellubiosis E-98167 |
| | | | | L. reuterii E-92142 |
| | | | | L. paracasei E-93490 |
| B. fragilis | BfraF, | 30-45% | 70 V, 960 mins. | B. caccae DSM 19024 |
| | BfraR-GC | | | B. uniformis DSM 6597 |
| | | | | B. eggerthii DSM 20697 |
| EREC | CcocF, | 40-58% | 70 V, 960 mins | L. multipara DSM 3073 |
| | CcocR-GC | | | A. cacae DSM 14662 |
| | | | | D. longicatena DSM 13814 |
| | | | | R. productus DSM 2950 |
| C. leptum | CleptF, | 30-53% | 70 V, 960 mins | F. prausnitzii DSM 17677 |
| | CleptR3-GC | | | C. methylpentosum DSM 5476 |
| | | | | R. albus DSM 20455 |
| | | | | C. leptum DSM 753 |
| | | | | E. siraeum DSM 15702 |
| | | | | C. viridae DSM 6836 |

*Primer sequences are in Table 2

### Example 1

Secretor status was determined from the blood samples by using an agglutination assay. Secretor status was determined from 59 individual and 48 were secretors and seven were non-secretors. The secretor status of four samples could not be determined; they were excluded from the further analyses.

### Example 2

In universal DGGE analysis of dominant intestinal bacteria, several genotypes occurred statistically significantly more often or with a higher intensity in the non-secretor samples than in the secretor samples. All genotypes were 2 to 3.6 times more frequently detected in the non-secretor in comparison to secretor samples. The genotypes can be identified by the band positions on universal DGGE gel corresponding the band positions 25.30%, 26.40%, 50.40% and 56.80%. The band positions, genotypes, which differed between non-secretor and secretor individuals and their detection frequencies, are shown in Table 3.

TABLE 3
Statistically significant differences on band intensities between
non-secretor (NSS) and secretor (SS) samples as determined by
universal-DGGE (n = 55, NSS = 7, SS = 48). Statistical
tests, ANOVA (ANO) and Kruskal-Wallis (KW) were based on band
intensity matrix and Fisher's exact test (F) was based on presence/
absence-matrix of the bands

| | | | | | | Mean |
|---|---|---|---|---|---|---|
| | | | | | | band |
| | | | | number | number | intensity |
| Geno- | | | | in NSS | in SS | in NSS/ |
| type | Test | p-value | # of hits | (%) | (% | in SS |
| 25.30% | ANO/KW | 0.03/0.05 | 18 (31) | 4 (57) | 14 (29) | 13/10 |
| 26.40% | ANO/KW | 0.002/0.02 | 4 (7) | 1 (14) | 3 (6) | 22/8 |
| 50.40% | ANO | 0.03 | 6 (10) | 2 (29) | 4 (8) | 18/10 |
| 56.80% | KW/F | 0.006/0.01 | 10 (17) | 4 (57) | 6 (12) | 17/25 |

### Example 3

A genotype belonging to Eubacterium rectale-Clostridium coccoides-group (EREC) and corresponding band position 60.0% in EREC-DGGE gels was clearly more common in non-secretor than in secretor samples. The genotype was more than seven times more common in the samples from non-secretor individuals than in the samples of secretor individuals. The results are shown in Table 4.

TABLE 4
Statistically significant differences on band intensities between
non-secretor (NSS) and secretor (SS) samples as determined by
EREC-DGGE (n = 55, NSS = 7, SS = 48). Statistical tests,
ANOVA (ANO) and Kruskal-Wallis (KW) were based on band
intensity matrix and Fisher's exact test (F) was based on presence/
absence-matrix of the bands

| | | | | | | Mean |
|---|---|---|---|---|---|---|
| | | | | | | band |
| | | p-value | | | number | intensity |
| Geno- | | (ANO/ | # of | number in | in SS | in NSS/ |
| type | Test | KW/F) | hits | NSS (%) | (%) | in SS |
| 60.00% | ANO/ | 0.00002/ | 6 (10) | 3 (43) | 3 (6) | 30/11 |
| | KW/F | 0.0006/ | | | | |
| | | 0.04 | | | | |

### Example 4

Five genotypes of Bacteroides fragilis group were statistically significantly more common or more abundant in the non-secretor samples than in secretor samples. The genotype band position 23.80, as indicated by the controls, referred to Bacteroides uniformis strain DSM6597; this genotype was three times more common in the non-secretor samples than in the secretor samples. Other genotypes corresponded band positions 4.80%, 10.20%, 38.70%, and 41.10%. These band positions were also three times more commonly detected in the non-secretor than in secretor samples, except genotypes related to band positions 10.20% and 38.70%. Band positions 10.20% and 38.70% were equally common in the non-secretor and secretor samples, but the band intensity (i.e. abundance) was over two times higher in the non-secretor than in secretor samples. The results are shown in Table 5.

TABLE 5
Statistically significant differences on band intensities between
non-secretor and secretor samples as determined by B. fragilis group
DGGE (n = 55, NSS = 7, SS = 48). Statistical tests, ANOVA
(ANO) and Kruskal-Wallis (KW) were based on the band intensity
matrix and Fisher's exact test (F) was based on presence/absence-
matrix of the bands

| | | | | | | Mean |
|---|---|---|---|---|---|---|
| | | | | | | band |
| | | p-value | | number | number | intensity |
| | | (ANO/ | | in NSS | in SS | in NSS/ |
| Genotype | test | KW/F) | # of hits | (%) | (%) | in SS |
| 4.80% | KW | 0.04 | 6 (10) | 2 (29) | 4 (8) | 54/61 |
| 10.20% | ANO | 0.004 | 29 (49) | 4 (57) | 25 (52) | 93/35 |
| 23.80% | ANO/ | 0.0004/ | 13 (22) | 4 (57) | 9 (19) | 62/32 |
| | KW/F | 0.005/ | | | | |
| | | 0.03 | | | | |
| 38.70% | ANO | 0.02 | 24 (41) | 3 (43) | 21 (44) | 96/16 |
| 41.10% | ANO | 0.007 | 7 (12) | 2 (29) | 5 (10) | 53/39 |

### Example 5

Seven genotypes belonging to Clostridium leptum group were more common or abundant in the non-secretor samples than in secretor samples. The band positions corresponding to these genotypes are listed in Table 6. The genotype in band position 36.10% was slightly more common in the non-secretors in comparison to the secretors, but this genotype was 3.8 times more abundant as measured by band intensity in the non-secretors. The results are shown in table 6.

TABLE 6
Statistically significant differences on band intensities between
non-secretor and secretor samples as determined by C. leptum
DGGE (n = 55, NSS = 7, SS = 48). Statistical tests, ANOVA
(ANO) and Kruskal-Wallis (KW) were based on band intensity matrix
and Fisher's exact test (F) was based on presence/absence-matrix
of the bands

| | | | | | | Mean |
|---|---|---|---|---|---|---|
| | | | | | | band |
| | | p-value | | number | | intensity |
| | | (ANO/ | # of | in NSS | number | in NSS/ |
| Genotype | test | KW) | hits | (%) | in SS (%) | inSS |
| 32.80% | KW | 0.003 | 6 (10) | 3 (43) | 3 (6) | 15/25 |
| 36.10% | ANO | 0.03 | 7 (10) | 1 (14) | 5 (10) | 54/11 |
| 43.00% | ANO | 0.007 | 16 (27) | 3 (43) | 13 (27) | 95/35 |
| 73.30% | ANO | 0.001 | 14 (24) | 3 (43) | 11 (23) | 25/19 |
| 79.10% | ANO | 0.01 | 6 (10) | 2 (29) | 4 (8) | 52/30 |
| 85.00% | ANO/ | 0.007/ | 15 (25) | 5 (71) | 10 (21) | 25/20 |
| | KW | 0.005 | | | | |
| 91.80% | ANO/ | 0.0008/ | 8 (14) | 3 (43) | 5 (10) | 52/15 |
| | KW | 0.01 | | | | |

### Example 6

In this example, the number of volunteers was increased to 71 by recruiting 12 new volunteers in addition to the 59 volunteers of examples 1-5. For these 71 volunteers, in addition to phenotyping, secretor status was genotyped by sequencing the coding exon of FUT2 as described in Silva et al. (Glycoconj J 2010, 27, 61-68) and Ferrer-Admetlla et al. (Mol Biol Evol 2009, 26, 1993-2003). Genotyping of FUT2 exon allowed determination of secretor status for the Lewis negative individuals, whose phenotypic secretor status could not be determined. The DGGE analysis and data-analysis were performed as described above. Statistical analyses, Anova and Fisher's exact test, were computed with statistical programming language R, version 2.10.1.

In the enlarged dataset (n=71), 57 individuals represented secretors and 14 represented non-secretors.

The analysis of the enlarged dataset with DGGE revealed that the incidence and/or band intensity of several genotypes (i.e. DGGE band positions) were significantly different between the groups. With exception of two genotypes in C. leptum groups and one genotype in EREC group, all the genotypes were more commonly detected in non-secretor individuals than secretor individuals. This confirmed the results of the previous examples 1-5, that several microbial genotypes are associated to the secretor status of the host.

The results are shown in table 7.

TABLE 7
The band positions of dominant bacteria, C. leptum, B. fragilis,
EREC, and Lactobacillus groups and the incidence of bands in secretor
(14) and non-secretor samples (57) analysed by PCR-DGGE. Only the
bands with a statistically significant difference between the secretors
(SS) and non-secretors (NSS) are shown.

| | | | Fisher's | in | in | |
|---|---|---|---|---|---|---|
| | Band | | exact | NSS, | SS, | # of |
| Group | position | ANOVA | test | % | % | hits |
| C. leptum | 15.40% | 0.03 | | 36% | 18% | 15 |
| C. leptum | 20.50% | 0.02 | | 29% | 14% | 12 |
| C. leptum | 27.80% | 0.0004 | 0.004 | 36% | 5% | 8 |
| C. leptum | 32.80% | 0.002 | 0.04 | 21% | 4% | 5 |
| C. leptum | 37.30% | 0.02 | 0.04 | 21% | 4% | 5 |
| C. leptum | 42.10% | 0.04 | | 29% | 21% | 16 |
| C. leptum | 46.50% | 0.03 | | 21% | 13% | 10 |
| C. leptum | 49.10% | | 0.03 | 50% | 77% | 50 |
| C. leptum | 56.20% | 0.02 | | 14% | 18% | 12 |
| C. leptum | 61.80% | 0.04 | 0.03 | 50% | 25% | 21 |
| C. leptum | 73.20% | 0.05 | | 36% | 23% | 18 |
| C. leptum | 85.0% | 0.02 | 0.007 | 71% | 29% | 26 |
| C. leptum | 91.80% | 0.04 | | 29% | 13% | 11 |
| B. fragilis | 36.0% | 0.02 | | 15% | 5% | 5 |
| B. fragilis | 38.60% | 0.009 | | 38% | 30% | 22 |
| EREC | 35.3% | 0.02 | | 29% | 11% | 10 |
| EREC | 60.0% | 0.001 | | 21% | 5% | 6 |
| EREC | 62.60% | 0.04 | | 14% | 33% | 21 |
| EREC | 69.0% | 0.002 | | 71% | 46% | 36 |
| Dominant bacteria | 25.20% | 0.02 | 0.05 | 43% | 28% | 22 |
| Dominant bacteria | 39.00% | 0.004 | 0.01 | 36% | 11% | 11 |
| Dominant bacteria | 42.40% | 0.02 | 0.07 | 29% | 9% | 9 |
| Dominant bacteria | 47.00% | 0.05 | | 21% | 7% | 7 |
| Dominant bacteria | 50.50% | 0.001 | 0.01 | 29% | 4% | 6 |
| Dominant bacteria | 56.60% | | 0.0005 | 64% | 14% | 17 |
| Dominant bacteria | 60.20% | 0.01 | | 36% | 25% | 19 |

### Example 7

The isolation of the bacteria representing a specific PCR-DGGE genotype (see Table 6) can be performed as follows. A faecal sample is cultured on non-selective or selective culture media in the conditions supporting the growth of the targeted bacterial group. In the present study Brucella blood agar (BRU) or reinforced clostridial agar with chinablue and horse blood (RCBA) was used for the cultivation of the anaerobic bacteria e.g. clostridia and their close relatives. In addition several selective culture media e.g. Bacteroides Bile esculin (BBE) for the isolation of Bacteroides spp. and its close relatives, Beerens and Raffinose bifidobacterium (RB) for bifidobacteria, and Rogosa and LAMVAB for lactobacilli, respectively, were used. All plates were incubated in anaerobic conditions except for those for lactobacilli cultures which were cultured either in anaerobic or microaerophilic conditions. Bacterial isolates were sub-cultured from the culture plates and identified to the species level by 16S rDNA sequencing. Selected isolates were further characterised by DGGE in parallel to a known sample with a particular DGGE band position to identify the strains underlying the genotype.

In addition to the direct culturing of the samples culturing was performed after pre-treatment with the TNO TIM-1 model, which mimics the conditions in the upper GI-tract. For the pre-treatment faecal slurries acquired by mixing faeces with artificial saliva and sterile water were used as input for the TIM-1 model. The faecal slurries were prepared from pooled non-secretor samples (n=11; total 12.1 g faeces) and secretor samples (n=11; total 9.8 g of faeces) were used. In TNO TIM-1 model T1/2 for emptying the gastric content was set to 20 min, pH change from pH 2.0 to 1.7 in 30 min and level of gastric secretion on 20%. The gastric content was passed into the duodenal compartment, where it was neutralized to pH 6.4, and bile and pancreatin were added, followed passage (time 10 minutes) into the jejunum compartment and into the ileum compartment. In each compartment the physiological concentrations of bile salts, pancreatic enzymes and electrolytes simulated in combination with an average physiological passage through the small intestine. The samples were collected from after 120-180, 180-240 and 240-300 mins treatment. Samples were collected from faecal slurries before the TIM-1 treatment (intake samples) and after the treatments. Dilution series of collected samples were plated in duplicate on applied culturing media and incubated for 72 hours at 37° C.

The survival of anaerobic bacteria (RCBA) from secretor pool was at least 10 times higher (25% vs 2.5%) than the survival of anaerobic bacteria from non-secretor pool (see Table 8). Similarly, the Bacteroides population (BBE) in the secretor group showed a higher survival rate in the secretor sampies than in non-secretor samples (1% vs 0%). Thus the population of anaerobic bacteria in non-secretor individuals was less tolerant for the harsh conditions of TNO TIM-1 model, mimicking environments in the stomach and small intestine. These results show that cultivable anaerobic population in non-secretor individuals differed from that in secretor individuals, indicating differences not only in the species composition but also in the functional characteristics of the bacterial populations.

TABLE 8
The survival of anaerobic bacteria from pooled faecal samples of
secretor and non-secretor individuals in the TIM-1 model (upper
gastroin-testinal tract conditions). Viability was determined by
plate count culturing using RCBA and BBE media.

| | Secretor pool | | Non-secretor pool | |
|---|---|---|---|---|
| | RCBA | BBE | BCBA | BBE |
| Intake, total cfu in | 2.3E+10 | 3.4E+08 | 2.9E+10 | 2.2E+09 |
| sample | | | | |
| Total survival, total cfu | 5.8E+09 | 3.5E+06 | 7.3E+08 | 0 |
| in samples | | | | |
| % survival | 25 | 1 | 2.5 | 0 |

### Example 8

The Human Intestinal Tract (HIT) chip (Rajilic-Stojanovic et al. 2009, Environ Microbiol 11 (7): 1736-1751) was applied for more detailed investigation of the microbiota in non-secretor and secretor individuals. HITchip contains approximately 5000 nucleotide probes targeting over 1000 phylotypes of bacteria colonising the human gut. HITChip analysis were performed as described in Rajilic-Stojanovic et al. 2009 for DNA (extracted from 1 g of DNA by the Apajalahti et al. 1998 method) samples of 12 non-secretor and 12 secretor individuals. The secretor subjects included were matched for non-secretors regarding to ABO blood group, age, and sex. The data was normalised and analysed in R using within-array spatial normalization and quality control as described in Rajilic-Stojanovic et al. 2009. On top of that between-array normalization was performed with quantile normalization. The differences for each bacterial group between the sample groups were studied with linear models and ANOVA-tests, transforming the array intensities into logarithmic scale first.

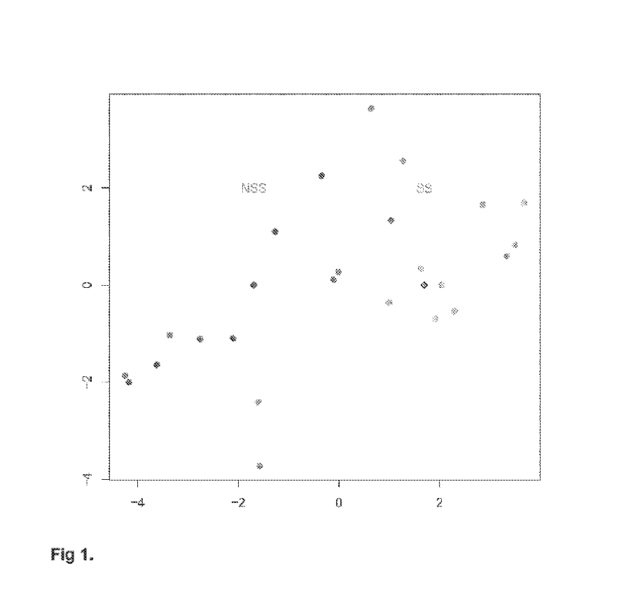

The non-secretor and secretor samples were clustered separately in the redundancy analysis (RDA), which was based on relative abundances of level 1 (species-like level) taxa. This indicates that the faecal microbiota structure of secretor individuals differed from the microbiota structure of non-secretor individuals, see FIG. 1.

Relative abundance, as determined by intensities of the hybridisation signals, of 35 taxa (species-like level 1) belonging to Bacteroides, EREC and C. leptum groups were significantly different (as tested by ANOVA) between non-secretor and secretor individuals. Of the taxa differing between non-secretor and secretor individuals, the taxa of Bacteroides were more abundant in secretor in comparison to non-secretor. Whereas the taxa of Faecalibacter prausnitzii et rel., Clostridium sphenoides et rel., and Clostridium symbiosum et rel. were more abundant in the non-secretors than secretors. The response to secretor status and to the presence of ABO and Lewis b antigens was found to be taxon-specific (see table 9), e.g. in taxa belonging to R. obeum et rel. Ruminococcus productus was more abundant in secretors while uncultured human gut bacterium JW1H7 and JW2A6 were more abundant in non-secretors; in Lachnospira pectinoschiza et rel Lachnospira pectinoschiza was more abundant in secretors while uncultured human gut bacterium JW1G3 was more abundant in non-secretors; and in Clostridium orbiscindens et rel. Clostridium orbiscindens was more abundant in secretors while uncultured bacterial clone Eldhufec272 was more abundant in non-secretors.

In addition, in classification level 3, the relative abundance of Clostridium cluster XV (p-value 0.04 in ANOVA) was significantly different between the non-secretors and secretors.

TABLE 9
The bacterial groups in level 1 (species-like level), whose relative abundances
were significantly different (p-value < 0.05 in ANOVA) between non-secretor
and secretor individuals.

| | | | Hybridisation | |
|---|---|---|---|---|
| Group, level 1 | Group, level 2 | Group, level 3 | signal | p-value |
| uncultured bacterium C706 | Allistipes et rel. | Bacteroides | SS > NSS | 0.02* |
| uncultured bacterium D080 | Allistipes et rel. | Bacteroides | SS > NSS | 0.02* |
| bacterium adhufec84 | Bacteroides splachnicus et rel. | Bacteroides | SS > NSS | 0.03* |
| uncultured bacterium MS86 | Bacteroides splachnicus et rel. | Bacteroides | SS > NSS | 0.02* |
| uncultured bacterium NG42 | Bacteroides splachnicus et rel. | Bacteroides | SS > NSS | 0.02* |
| uncultured bacterium NK71 | Bacteroides splachnicus et rel. | Bacteroides | SS > NSS | 0.02* |
| uncultured bacterium NK90 | Bacteroides splachnicus et rel. | Bacteroides | SS > NSS | 0.02* |
| uncultured bacterium NN42 | Bacteroides splachnicus et rel. | Bacteroides | SS > NSS | 0.02* |
| uncultured bacterium NP53 | Bacteroides splachnicus et rel. | Bacteroides | SS > NSS | 0.03* |
| bacterium adhufec77.25 | Tannerella et rel. | Bacteroides | SS > NSS | 0.04* |
| uncultured bacterium LF02 | Anaerotruncus colihominis et rel. | C. leptum (cluster IV) | NSS > SS | 0.03* |
| uncultured bacterium MH24 | Clostridium cellulosi et rel. | C. leptum (cluster IV) | SS > NSS | 0.04* |
| Clostridium orbiscindens | Clostridium orbiscindens et rel. | C. leptum (cluster IV) | SS > NSS | 0.02* |
| Uncultured bacterium clone Eldhufec272 | Clostridium orbiscindens et rel. | C. leptum (cluster IV) | NSS > SS | 0.03* |
| Uncultured bacterium clone Eldhufec255 | Faecalibacterium prausnitzii et rel. | C. leptum (cluster IV) | NSS > SS | 0.0008*** |
| Uncultured bacterium clone Eldhufec261 | Faecalibacterium prausnitzii et rel. | C. leptum (cluster IV) | NSS > SS | 0.004** |
| Uncultured bacterium clone Eldhufec282 | Faecalibacterium prausnitzii et rel. | C. leptum (cluster IV) | NSS > SS | 0.004** |
| Uncultured bacterium clone Eldhufec284 | Ruminococcus callidus et rel. | C. leptum (cluster IV) | NSS > SS | 0.03* |
| Bryantella formatexigens | Bryantella formatexigens et rel. | EREC (cluster XIVa) | NSS > SS | 0.04* |
| uncultured bacterium G075 | Clostridium colinum et rel. | EREC (cluster XIVa) | NSS > SS | 0.02* |
| bacterium adhufec382 | Clostridium sphenoides et rel. | EREC (cluster XIVa) | NSS > SS | 0.02* |
| Uncultured bacterium clone Eldhufec131 | Clostridium sphenoides et rel. | EREC (cluster XIVa) | NSS > SS | 0.001** |
| Clostridium bolteae | Clostridium symbiosum et rel. | EREC (cluster XIVa) | NSS > SS | 0.02* |
| uncultured bacterium B147 | Clostridium symbiosum et rel. | EREC (cluster XIVa) | NSS > SS | 0.03* |
| uncultured bacterium HuCC34 | Clostridium symbiosum et rel. | EREC (cluster XIVa) | NSS > SS | 0.04* |
| uncultured bacterium inhufecA-32 | Clostridium symbiosum et rel. | EREC (cluster XIVa) | NSS > SS | 0.02* |
| Butyrivibrio fibrisolvens | Eubacterium rectale et rel. | EREC (cluster XIVa) | SS > NSS | 0.04* |
| Lachnospira pectinoschiza | Lachnospira pectinoschiza et rel. | EREC (cluster XIVa) | SS > NSS | 0.04* |
| uncultured human gut bacterium JW1G3 | Lachnospira pectinoschiza et rel. | EREC (cluster XIVa) | NSS > SS | 0.02* |
| Ruminococcus productus | Ruminococcus obeum et rel. | EREC (cluster XIVa) | SS > NSS | 0.02* |
| uncultured human gut bacterium JW1H7 | Ruminococcus obeum et rel. | EREC (cluster XIVa) | NSS > SS | 0.03* |
| uncultured human gut bacterium JW2A6 | Ruminococcus obeum et rel. | EREC (cluster XIVa) | NSS > SS | 0.03* |

### Example 9

A same subset of 24 DNA samples (12 from non-secretor individuals and 12 secretor individuals), which was studied by HITChip in Example 8, was analysed by pyrosequencing the V1-V3 region of 16S rRNA gene. The 16S rRNA gene fragment was PCR amplified using universal primer pair (F28 5′-AGAGTTTGATCMTGGCTCAG-3′; 518R 5′-ATTACCGCGGCTGCTGG-3′). The F primer contained adaptor sequence (5′-CGTATCGCCTCCCTCGCGCCATCAG-3′) and 6 base-long barcode tag, and R primer contained adaptor sequence (5′-CTATGCGCCTTGCCAGCCCGCTCAG-3′). The barcode sequence was unique for each sample and provided by Institute of Biotechnology (University of Helsinki). PCR reaction mixture (25 μA was composed of 0.2 μM of each primer (Sigma-Aldrich, UK), 0.2 mM dNTP mixture, 1× Phusion HF buffer (Finnzymes, Finland), 0.5 U Phusion polymerase (Finnzymes, Finland), 3% DSMO and 1 μl DNA template diluted to the concentration of 20 ng/μl. The PCR amplification conditions were one cycle of 98° C. for 1 min, followed by 35 cycles of denaturation 98° C. for 10 s, annealing at 65° C. for 30 s and elongation at 72° C. for 10 s. All the samples were run in three replicates. The PCR products were quantified and pooled in equal amounts. Emulsion PCR was performed from the pool and 454 pyrosequencing was done on the Genome sequencer FLX Titanium (Roche) in Institute of Biotechnology (University of Helsinki, Finland).

The raw sequences (245 806) were trimmed using Mothur (v.1.19.0) software (Schloss et al. Appl Environ Microbiol 2009, 75, 7537-41). The sequences with averaged quality score >30, length over 300 bases, exact matches to barcode tags and forward primer, no ambiguous bases, no homopolymers longer than 8 bp, and non-chimeric according to Chimera Slayer implemented in Mothur were included to the analysis (127 352 sequences, 52%). One non-secretor sample was excluded from analysis as an outlier sample. The high-quality sequences were binned to samples according the barcode tags, and into operational taxonomic units (OTUs) using threshold distance 0.03. Distance matrix of samples was calculated in Mothur using Jaccard Index and Bray-Curtis index. Jaccard Index accounts the presence/absence of OTUs and describes dissimilarity in microbial community membership. Bray-Curtis index accounts also abundance of each OTUs and describes the dissimilarity of community structures. Microbial community membership and structure between non-secretor and secretor samples was compared by AMOVA (analysis of molecular variance) with 1000 randomisations as implemented in Mothur.

The microbial community structure (calculated by Bray-Curtis index) differed between the non-secretor and secretor samples in analysis of AMOVA (p=0.027), whereas the membership of OTUs between non-secretor and secretor samples did not (see table 10). These results increased the amount of evidence that the non-secretor and secretor individuals have significant differences in their intestinal microbiota community, and that the secretor status is one of the significant host genotypic features that explain the inter-individual variation of the microbiota in humans.

TABLE 10
Statistics of AMOVA test measuring the difference in community
structure (Bray-Curtis) and membership (Jaccard) between non-
secretor (n = 11) and secretor samples (n = 12).

| | MS among and | | |
|---|---|---|---|
| Test | within populations | Fs statistics | P-value |
| AMOVA, Bray-Curtis | 0.41/0.29 | 1.42 | 0.027* |
| AMOVA, Jaccard | 0.46/0.44 | 1.03 | 0.129^{ns} |

^{ns}= non-significant

1.-23. (canceled)

24. A non-secretor/secretor genotype based microbial composition, wherein the composition is tailored based on the spectrum of bacteria found in the mucosal tissue of at least one individual with non-secretor or secretor blood group phenotype.

25. The microbial composition according to claim 24, wherein the composition comprises at least one of the strains having any of the following genotypes:
band position 25.30%, 26.40%, 39.00%, 42.40%, 47.00%, 50.40%, 56.80% or 60.20% as defined by universal-DGGE analysis; or
band position 35.30%, 60.00%, 62.60% or 69.00% as defined by Eubacterium rectale-Clostridium coccoides-group (EREC)-DGGE analysis; or
band position 4.80%, 10.20%, 23.80%, 36.00%, 38.70% or 41.10% as defined by Bacteroides-DGGE analysis; or
band position 15.40%, 20.50%, 27.80%, 32.80%, 36.10%, 37.30%, 42.10%, 43.00%, 46.10%, 56.20%, 61.80% 73.30%, 79.10%, 85.00% or 91.80%, as defined by Clostridium leptum-DGGE analysis.

26. The microbial composition of claim 24, wherein the composition is tailored based on the spectrum of bacteria found in the mucosal tissue of at least one individual with non-secretor blood group phenotype.

27. The microbial composition according to claim 26, wherein the composition comprises at least one of the strains having any of the following genotypes:
band position 25.30%, 26.40%, 39.00%, 42.40%, 47.00%, 50.40%, 56.80% or 60.20% as defined by universal-DGGE analysis; or
band position 35.30%, 60.0% or 69.00% as defined by Eubacterium rectale-Clostridium coccoides-group (EREC)-DGGE analysis; or
band position 4.80%, 10.20%, 23.80%, 36.00%, 38.70% or 41.10% as defined by Bacteroides-DGGE analysis; or
band position 15.40%, 20.50%, 27.80%, 32.80%, 36.10%, 37.30%, 42.10%, 43.00%, 46.10%, 61.80%, 73.30%, 79.10%, 85.00% or 91.80% as defined by Clostridium leptum-DGGE analysis.

28. The microbial composition according to claim 27, wherein the composition comprises two or more of the specified strains.

29. The microbial composition according to claim 27, wherein the composition comprises at least one of the strains having any of the following bacterial genotypes
a) band position 25.30%, 26.40%, 50.40% or 56.80% as defined by universal-DGGE analysis; or
b) band position 60.0% as defined by Eubacterium rectale-Clostridium coccoides-group (EREC)-DGGE analysis; or
c) band position 4.80%, 10.20%, 23.80%, 38.70%, or 41.10% as defined by Bacteroides-DGGE analysis; or
d) band position 32.80%, 36.10%, 43.00%, 73.30%, 79.10%, 85.00%, or 91.80% as defined by Clostridium leptum-DGGE analysis.

30. The microbial composition according to claim 29, wherein the composition comprises two or more of the specified strains.

31. The microbial composition of claim 24, wherein the composition is tailored based on the spectrum of bacteria found in the mucosal tissue of at least one individual with secretor blood group phenotype.

32. The microbial composition according to claim 31, wherein the composition comprises at least one of the strains having any of the following genotypes:
band position 62.60% as defined by Eubacterium rectale-Clostridium coccoides-group (EREC)-DGGE analysis; or
band position 56.20% as defined by Clostridium leptum-DGGE analysis.

33. The microbial composition according to claim 31, wherein the composition comprises the strains having any of the following genotypes:
band position 62.60% as defined by Eubacterium rectale-Clostridium coccoides-group (EREC)-DGGE analysis; and
band position 56.20% as defined by Clostridium leptum-DGGE analysis.

34. The microbial composition according to claim 24, wherein the composition is tailored based on the spectrum of non-bifidobacterial bacteria found in the mucosal tissue of at least one individual with non-secretor or secretor blood group phenotype.

35. The microbial composition according to claim 24, wherein the said composition additionally includes at least one prebiotic agent.

36. The microbial composition according to claim 24 for treating and/or preventing inflammatory bowel disease, diarrhoea, respiratory tract infections, irritable bowel syndrome, atopy/allergy, celiac disease and/or disturbed balance of mucosal microbiota followed by stem cell transplantation and/or subsequent graft-versus-host disease.

37. A method of tailoring a microbial composition based on the spectrum of bacteria found in the mucosal tissue of at least one individual with non-secretor or secretor blood group phenotype.

38. The method according to claim 37, wherein the method comprises steps:
determining secretor/non-secretor genotype of an individual,
determining the typical mucosal bacterial repertoire of the secretor/non-secretor type,
optionally manufacturing a microbial composition based on the determined bacterial repertoire.

39. A method of assessing the need of an individual for optimized microbial supplementation or predicting the microbial composition of the gut microbiota of the said individual by determining the secretor/non secretor blood group status of the individual.

40. The method according to claim 39, wherein the predicted microbial composition is related to at least one of the bacterial group of the list: Bacteroides fragilis group, Clostridium leptum group, and/or Eubacterium rectale-Clostridium coccoides-group.

41. A method for determining the balance of gut microbiota of an individual, the method comprising the steps of:
determining secretor/non-secretor genotype of an individual,
determining the composition of gut microbiota of the said individual,
comparing the composition of the gut microbiota of the said individual to the typical composition of gut microbiota according to the secretor/non-secretor genotype.

42. A method of estimating a dose of microbial supplementation needed for a desired effect in an individual, or augmenting stabilisation of mucosal microbiota of an individual in disorders related to, or after treatments leading to unbalance of mucosal microbiota by determining the secretor/non secretor blood group status of the individual.


SEQUENCE LISTING (14 sequences)

SEQ ID NO: 1 - DNA, 56 bp, Artificial Sequence
  Primer
cgcccggggcgcgccccgggcggggcgggggcacggggggaacgcgaagaacctta

SEQ ID NO: 2 - DNA, 17 bp, Artificial Sequence
  Primer
cggtgtgtacaagaccc

SEQ ID NO: 3 - DNA, 19 bp, Artificial Sequence
  Primer
agcagtagggaatcttcca

SEQ ID NO: 4 - DNA, 58 bp, Artificial Sequence
  Primer
cgcccgccgcgccccgcgcccggcccgccgcccccgccccattycaccgctacacatg

SEQ ID NO: 5 - DNA, 20 bp, Artificial Sequence
  Primer
aaatgacggtacctgactaa

SEQ ID NO: 6 - DNA, 62 bp, Artificial Sequence
  Primer
cgcccggggcgcgccccgggcggggcgggggcacggggggctttgagtttcattcttgcgaa

SEQ ID NO: 7 - DNA, 21 bp, Artificial Sequence
  Primer
atagcctttcgaaagraagat

SEQ ID NO: 8 - DNA, 61 bp, Artificial Sequence
  Primer
cgcccggggcgcgccccgggcggggcgggggcacggggggccagtatcaactgcaatttta

SEQ ID NO: 9 - DNA, 16 bp, Artificial Sequence
  Primer
gcacaagcagtggagt

SEQ ID NO: 10 - DNA, 58 bp, Artificial Sequence
  Primer
cgcccggggcgcgccccgggcggggcgggggcacggggggcttcctccgttttgtcaa

SEQ ID NO: 11 - DNA, 20 bp, Artificial Sequence
  Primer
agagtttgatcmtggctcag

SEQ ID NO: 12 - DNA, 17 bp, Artificial Sequence
  Primer
attaccgcggctgctgg

SEQ ID NO: 13 - DNA, 25 bp, Artificial Sequence
  Primer
cgtatcgcctccctcgcgccatcag

SEQ ID NO: 14 - DNA, 25 bp, Artificial Sequence
  Primer
ctatgcgccttgccagcccgctcag